(19) United States
(12) Patent Application Publication
(10) Pub. No.: US 2022/0382938 A1
(43) Pub. Date: Dec. 1, 2022

(54) Material Design System and Material Design Method

(71) Applicant: Hitachi, Ltd, Chiyoda-ku, Tokyo (JP)

(72) Inventors: Hiroshi ITO, Tokyo (JP); Shigenori MATSUMOTO, Tokyo (JP)

(21) Appl. No.: 17/776,081

(22) PCT Filed: Sep. 8, 2020
(86) PCT No.: PCT/JP2020/033912

§ 371 Date: May 11, 2022

(30) Foreign Application Priority Data
Nov. 15, 2019 (JP) 2019-206643

(51) Int. Cl. G06F 30/27; G16C 10/00; G06F 30/23; G16C 20/70

(52) U.S. Cl. CPC G06F 30/27; G16C 10/00; G06F 30/23; G16C 20/70

(57) ABSTRACT

An object of the present invention is to provide a material design system and a material design method, which each reduce computational complexity and enable efficient material design. A material design system according to the present invention, which designs a material that can achieve a desired material function, includes an input device receiving the desired material function, and an arithmetic unit calculating a constitutional material. The arithmetic unit includes a structure calculation part that calculates a molecular characteristic amount meeting the desired material function using a method that can express molecular characteristic amount exhibited by a set of two or more atoms or molecules, and a molecular calculation part that calculates the constituent material, which achieves the molecular characteristic amount calculated by the structure calculation part, using a method that can express a molecule itself.

### TECHNICAL FIELD

The present invention relates to a material design system and a material design method.

### BACKGROUND ART

Design of materials, such as inorganic materials including ceramics and metals, organic materials including resins, and inorganic-organic composite materials typified by materials for biotechnology, is performed using various methods such as experiments, analysis, and simulation. Digital techniques are recently increasingly used in design of inorganic or organic materials. A technique using data or information related to material is now used in addition to a technique for evaluating a material by simulation using a computer.

As exemplary material design of an inorganic material, patent literature 1 discloses a method for seeking a new crystal structure with a combination of first principle calculation and a genetic algorithm. A crystal structure model to be an execution object of the first principle calculation is determined using the genetic algorithm, and thus material search is achieved with calculation cost reduced.

As exemplary material design of an organic material using simulation, patent literature 2 discloses that a coarse-grained model of a polymer material is obtained using a predetermined parameter group, and the coarse-grained model is used to predict at least one property of the polymer material by simulation based on molecular dynamics.

### CITATION LIST

### Patent Literature

[PTL1] Japanese Unexamined Patent Application Publication No. 2018-10428.
[PTL2] Japanese Unexamined Patent Application Publication No. 2006-338449.

### SUMMARY OF INVENTION

### Technical Problem

Patent literature 1 discloses that the most stable structure of crystal of molecules of an inorganic compound constituting a material is estimated by a technique using the genetic algorithm. However, such a method does not clarify a relationship between a stable structure of crystal and a material function of an inorganic compound having that crystal structure. If the most stable structure of crystal of each inorganic compound is beforehand obtained to estimate a material function for that crystal structure, the most stable structure of crystal of every single inorganic compound must be obtained, anxiously causing large computational complexity.

Patent literature 2 discloses a method for designing a polymer material suitable for a desired application by predicting influence of a predetermined parameter group (including chain length, flexibility, and density) on properties. Patent literature 2 further discloses a method for designing a real polymer corresponding to a combination of preferred parameters, in which various factors of existing polymer materials, such as compositions and manufacturing conditions, are optimized to produce a polymer material well suited to the combination of the preferred parameters. However, patent literature 2 does not disclose a specific method for optimizing the parameter group, the composition of the real material, the manufacturing condition, and the like. If a person having expertise needs to select the preferred parameter group of the polymer material or optimize the parameter group by try and error, the material design based on patent literature 2 is not adequately processed on a system, and is thus probably insufficient.

Hence, it is desired to reduce restrictions on a user or an execution resource and improve efficiency of material design. An object of the invention is therefore to provide a material design system and a material design method, which each reduce computational complexity and enable efficient material design.

### Solution to Problem

To solve the above problem, a material design system according to the present invention includes an input device receiving a desired material function and an arithmetic unit calculating a constitutional material. The arithmetic unit includes a structure calculation part that calculates a molecular characteristic amount meeting the desired material function using a method that can express molecular characteristic amount exhibited by a set of two or more atoms or molecules, and a molecular calculation part that calculates the constituent material, which achieves the molecular characteristic amount calculated by the structure calculation part, using a method that can express a molecule itself.

### Advantageous Effects of Invention

According to the invention, it is possible to provide a material design system and a material design method, which each enable efficient material design. Other problems, configurations, and effects are clarified by the following embodiment.

### BRIEF DESCRIPTION OF DRAWINGS

FIG. 1 is a schematic diagram illustrating an exemplary configuration of a material design system according to one embodiment of the present invention.

FIG. 2 is a schematic diagram illustrating an exemplary input screen according to one embodiment of the invention.

FIG. 3 is a schematic diagram illustrating another exemplary input screen according to one embodiment of the invention.

FIG. 4 is a schematic diagram illustrating an exemplary relationship between a material function, a molecular characteristic amount, and a constituent material.

FIG. 5 is a schematic diagram illustrating an exemplary set of memory values.

FIG. 6 is a schematic diagram illustrating a DIKW pyramid.

FIG. 7 is a schematic diagram illustrating an exemplary data structure of memory values stored in a memory device.

FIG. 8A illustrates an exemplary relationship between nodes.

FIG. 8B illustrates another exemplary relationship between nodes.

FIG. 9 illustrates exemplary relationships between nodes 2A and 3A, between nodes 2A and 3B, and between nodes 2A and 3C.

FIG. 10 is a schematic diagram illustrating an exemplary set of memory values using properties.

FIG. 11 is a matrix diagram illustrating an exemplary relationship between nodes according to one embodiment of the invention.

FIG. 12 is a matrix diagram illustrating another exemplary relationship between nodes according to one embodiment of the invention.

FIG. 13 is a schematic diagram illustrating an exemplary configuration of a material design system using external devices.

FIG. 14 is a flowchart illustrating an exemplary processing procedure of a material design system according to one embodiment of the invention.

FIG. 15 is a schematic diagram illustrating in a simplified manner an exemplary relationship between the material function, the molecular characteristic amount, and the constituent material.

FIG. 16 is a schematic diagram illustrating an exemplary relationship between the material function, the molecular characteristic amount, and the constituent material with hierarchical categorization.

FIG. 17 is a flowchart illustrating an exemplary processing procedure of detailed investigation of a correlation between a material function and molecular characteristic amount in step 13 of FIG. 14.

FIG. 18 is a flowchart illustrating an exemplary processing procedure of detailed investigation of a correlation between molecular characteristic amount and a constituent material.

FIG. 19 is a schematic diagram illustrating an exemplary relationship for a polymeric material between a material function, molecular characteristic amount, and a constituent material.

FIG. 20A is a schematic diagram illustrating an exemplary spherulite of a polymeric material.

FIG. 20B is a schematic diagram illustrating another exemplary spherulite of a polymeric material.

FIG. 21A illustrates an exemplary correlation between a node of spherulite size and a node of tensile strength.

FIG. 21B illustrates an exemplary correlation between a node of the degree of orientation and a node of spherulite size.

FIG. 21C illustrates an exemplary correlation between a node of interaction and a node of the degree of orientation.

FIG. 21D illustrates an exemplary correlation between a node of a functional group type and a node of interaction.

FIG. 22A is a schematic diagram as a base of the investigation of the relationship between the tensile strength and the spherulite size in FIG. 21A.

FIG. 22B is a schematic diagram as a base of the investigation of the relationship between the spherulite size and the degree of orientation in FIG. 21B.

FIG. 22C is a schematic diagram as a base of the investigation of the relationship between the degree of orientation and the interaction in FIG. 21C.

FIG. 22D is a schematic diagram as a base of the investigation of the relationship between the interaction and the functional group type in FIG. 21D.

### DESCRIPTION OF EMBODIMENTS

Hereinafter, an embodiment of the invention are described in detail with reference to drawings. The invention includes various modifications or alterations without being limited to the following embodiment. For example, a configuration of this embodiment can be subjected to addition, elimination, or substitution.

<Material Design System>

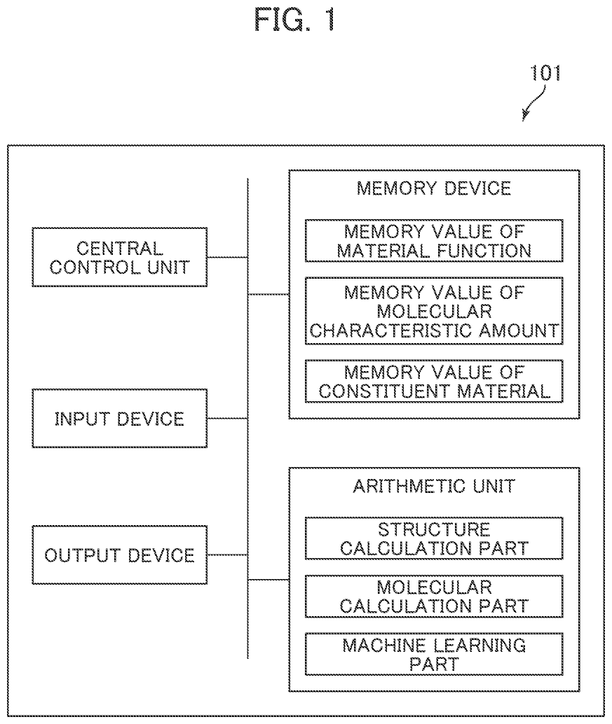

FIG. 1 is a schematic diagram illustrating an exemplary configuration of a material design system according to one embodiment of the present invention. A material design system 101 according to one embodiment of the invention includes an input device, an arithmetic unit, a memory device, an output device, and a central control unit. Such devices and units are interconnected together to allow data transfer. The respective devices or units are now described.

Examples of the input device include a mouse and a keyboard. The input device receives contents from a user. The input device receives not only a material function desired by a user but also information and data used for arithmetic operation in the arithmetic unit. The received contents specifically include a material function, molecular characteristic amount, a constituent material, and parameters of those.

The material function means a function corresponding to an application of a material. Examples of the material function include drying rate, oxidation rate (occurrence of rust), disinfection ability, detergency, heat insulation, soundproof ability, tensile strength, viscosity, adhesion, ignition speed, malleability, and ductility. The material function is directly linked to market value of a material.

The constituent material is definition of a material as a substance. Examples of the constituent material includes an element name, an electron state, a molecule name, a molecular structure, a molecular orbital, molecular weight, a functional group type, chemical bond, atomic arrangement, a crystal structure, a composition, and a plane direction. The constituent material specifically shows a material itself.

The molecular characteristic amount means physical properties, phenomena, properties, and the like exhibited by the constituent material, and is determined by a set of atoms or molecules. The molecular characteristic amount includes intramolecular characteristic amount and intermolecular characteristic amount. Characteristic amount exhibited by a set of two or more atoms is defined as the intramolecular characteristic amount. Examples of the intramolecular characteristic amount include bond length, molecular size, molecular rigidity, and polarization. Characteristic amount exhibited by a set of two or more molecules is defined as the intermolecular characteristic amount. Examples of the intermolecular characteristic amount include molecular ratio, interactive force, and chemical reactivity. For an inorganic material, while definition of a molecule is difficult, molecular characteristic amount exhibited by a set of two or more atoms is also assumed as the intramolecular characteristic amount herein. One or more pieces of the molecular characteristic amount describes items such as a principle, a mechanism, and a structure, through each of which the material function is enough to be exhibited by the constituent material, and describes additional items such as conditions for such items. Hence, when the material function, the molecular characteristic amount, the constituent material, and relations among them are sufficiently obtained, information is shown to be able to produce any material.

The parameters described herein refers to information and data such as conditions necessary for investigating the relations among the material function, the molecular characteristic amount, the constituent material, or the relations between the material functions, between the molecular characteristic amounts, and between the constituent materials. For example, in case of using simulation, the parameters include size of an analysis model, an ensemble condition, and numerical values of various control factors.

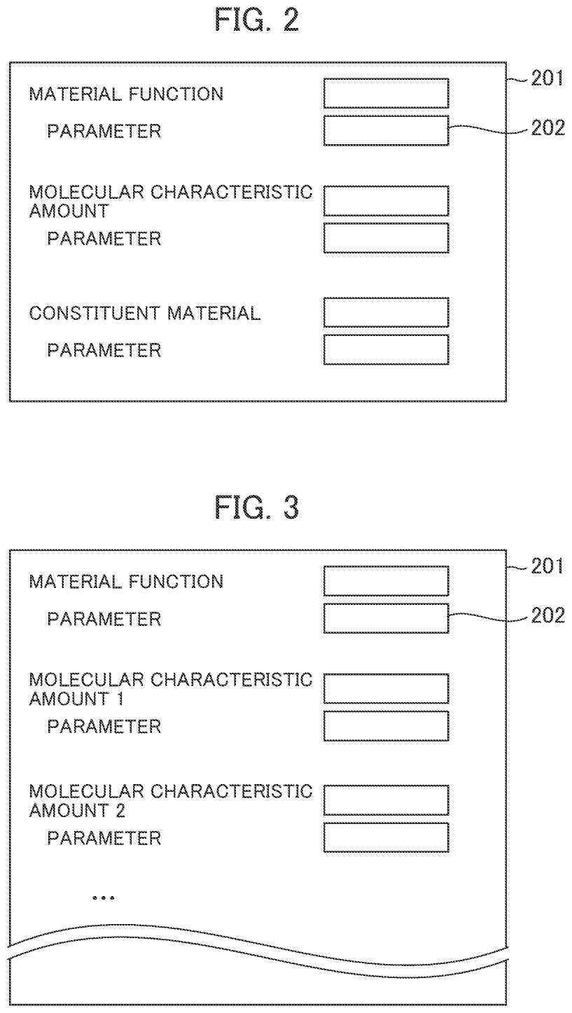

FIG. 2 is a schematic diagram illustrating an exemplary input screen according to one embodiment of the invention. An input screen 201 has entry fields 202 for the respective parameters of the material function, the molecular characteristic amount, and the constituent material. A user makes an entry in the entry field or uploads a file or the like, thereby the input device receives the input contents. FIG. 3 is a schematic diagram illustrating another exemplary input screen according to one embodiment of the invention. As illustrated in FIG. 3, an input mode may also be used so as to receive a plurality of molecular characteristic amounts.

The arithmetic unit includes, for example, a central processing unit (CPU) and software. As illustrated in FIG. 1, the arithmetic unit includes at least a structure calculation part and a molecular calculation part.

The structure calculation part calculates a molecular characteristic amount, which meets a desired material function received by the input device, with a method that can express the molecular characteristic amount. For example, the structure calculation part calculates the molecular characteristic amount meeting a desired material function by investigating a correlation between the material function and the molecular characteristic amount. Examples of arithmetic operation executable by the structure calculation part include an all-atom molecular dynamics method, a coarse-grained molecular dynamics method, a dissipative particle dynamics method, Monte Carlo method, Cellular Automaton, a particle method, a finite element method, a finite difference method, and a finite volume method. The above methods can each express a molecular characteristic amount.

The molecular calculation part calculates a constituent material, which achieves the molecular characteristic amount calculated by the structure calculation part, using a method that can express a molecule itself. For example, the molecular calculation part calculates the constituent material achieving the molecular characteristic amount calculated by the structure calculation part by investigating a correlation between the molecular characteristic amount and the constituent material. Examples of arithmetic operation executable by the molecular calculation part include the all-atom molecular dynamics method, an empirical molecular orbital method, a static first principle calculation method, and a first principle molecular dynamics method. A molecular orbital method or a density-functional approach can be used as the static first principle calculation method, and the Car-Parrinello method can be used for the first principle molecular dynamics method. The above methods can each express a molecule itself. A flow of processing by the arithmetic unit is described later.

The structure calculation part and the molecular calculation part may calculate the molecular characteristic amount and the constituent material, respectively, using experimental results or analysis results of literatures. For example, the structure calculation part or the molecular calculation part obtains experimental results or analysis results via the input device or an external network, and uses statistical analysis, machine learning operation, or literature search to calculate the molecular characteristic amount or the constituent material. The arithmetic unit may further include a machine learning part for machine learning operation (see FIG. 1).

The machine learning part performs machine learning operation on the experimental results, the analysis results, or the searched literatures. Examples of arithmetic operation executable by the machine learning part include a typical statistical method and deep learning. Typical, executable statistical methods include various examinations, principal component analysis, and regression analysis.

The arithmetic unit determines the significance of whether the molecular characteristic amount calculated by the structure calculation part meets a desired material function. If no significance is determined, the structure calculation part calculates again a molecular characteristic amount that meets the desired material function. The arithmetic unit further determines the significance of whether the constituent material calculated by the molecular calculation part meets the molecular characteristic amount calculated by the structure calculation part. If no significance is determined, the molecular calculation part calculates again a constituent material that achieves the molecular characteristic amount calculated by the structure calculation part.

A flow of processing by the arithmetic unit is described later.

The memory device includes a typical hard disc, for example. The memory device stores as memory values information and data received by the input device, and the material function, the molecular characteristic amount, and the constituent material obtained by the arithmetic unit. Specifically, the memory device stores as nodes the material function, the molecular characteristic amount, and the constituent material and stores as edges relations between the nodes based on the calculation result of the structure calculation part and the calculation result of the molecular calculation part.

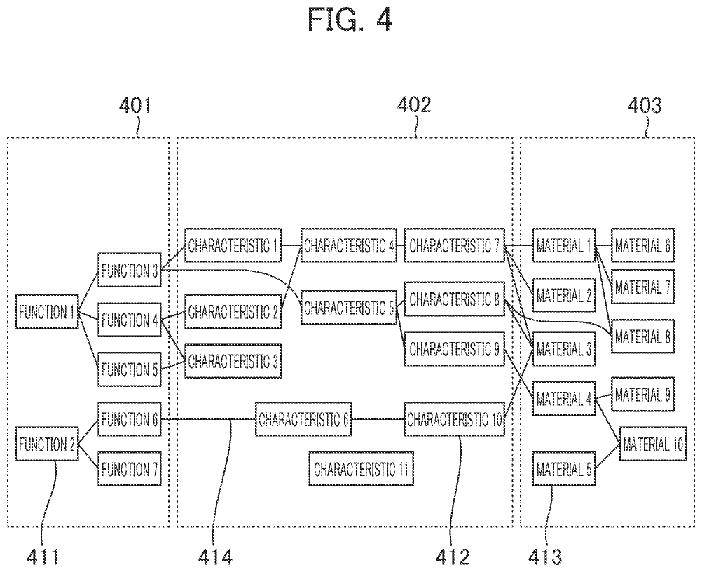

FIG. 4 illustrates an exemplary relationship between the material function, the molecular characteristic amount, and the constituent material. The respective items of the material functions, the molecular characteristic amounts, and the constituent materials are shown as nodes, and relations between the nodes are shown as edges. Each node is often expressed by words, but may be expressed in another form, for example, by a symbol, by a numerical formula, or by a picture. The edge is shown when some correlation exists between two nodes. The correlation includes a causation.

In this data structure, a plurality of nodes exist as typified by a material function node 411, a molecular characteristic amount node 412, and a constituent material node 413. The nodes are connected to each other by the edge 414. In addition, a material-function node group 401, a molecular-characteristic-amount node group 402, and a constituent-material node group 403 are shown. Each node belongs to any one of the material function node group 401, the molecular characteristic amount node group 402, and the constituent material node group 403. Nodes in each node group may be connected to each other by an edge. The nodes may be connected across the node group. Any number of nodes or edges is acceptable. Any number of edges may be connected to one node. Although the node can exist while being connected to no edge, each edge should be connected to one node at either end of the edge.

As described above, the nodes and the edges are used to indicate relations between the material function, the molecular characteristic amount, and the constituent material, which in turn indicates a type of the molecular characteristic amount with which an optional material function correlates, and indicates a type of the constituent material with which that molecular characteristic amount correlates. This allows indication of a noticeable molecular characteristic amount and a noticeable constituent material for a desired material function.

When the node and the edge, each being directly related to a focused material function, are prepared, feasibility of the material function can be predicted. When the node and the edge of the molecular characteristic amount or the constituent material, each being indirectly related to the focused material function, are prepared in addition to the node and edge directly related to the focused material function, means for achieving the material function, i.e., means for preparing the material can be clarified. This further makes it possible to predict or specify a lacking node and/or a lacking edge, i.e., a point to be investigated in material development. As a result, an execution plan of experiment, analysis, or simulation can be effectively made.

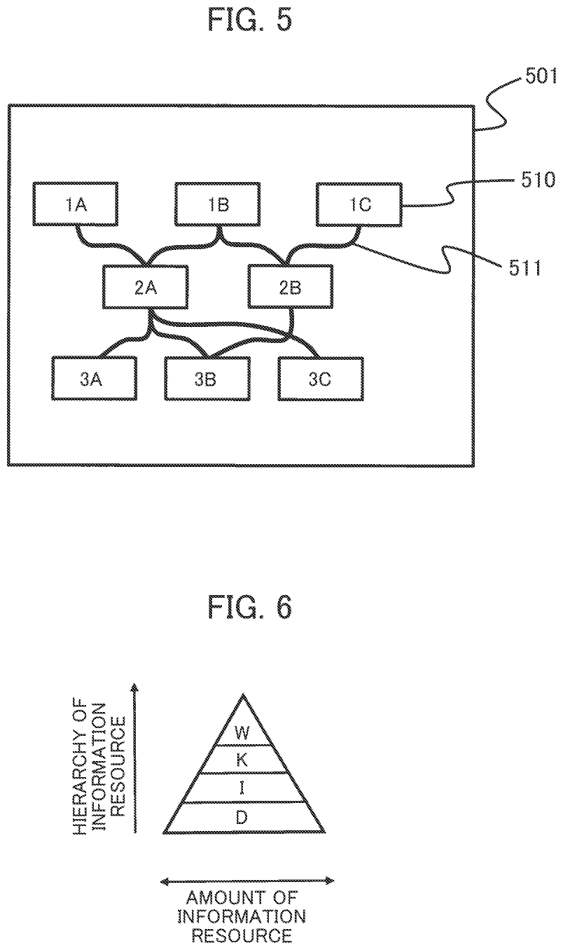

FIG. 5 is a schematic diagram illustrating an exemplary set of memory values. A set 501 of optional memory values is configured of node values 510 and edge values 511. The node values 510 indicates respective items of the material function, the molecular characteristic amount, and the constituent material. The edge values 511 may be expressed in any form, for example, by numerical values, by words, or by symbols, but are desirably expressed by numerical values. When a node value 510 is connected to a plurality of edge values 511, expression by numerical values makes it possible to express an edge value 511 having a relatively deep relation to the node value 510. Any number of node values 510 and any number of edge values 511 are acceptable. Any number of edge values 511 may be connected to one node. Although the node can exist while being connected to no edge, each edge should be connected to one node at either end of the edge.

Although any storage format may be used for storing the node values 510 or the edge values 511, a storage format of a graph structure type or a table is desirable. The storage format of the graph structure type enables fast search of a memory value when one memory value is extracted from among many node values 510 and many edge values 511 to search another node correlating with the memory value. In addition, the node values 510 are easily categorized or arranged based on the edge values 511. Further, since a relationship is easily shown diagrammatically, a user can efficiently understand the present system in inputting and outputting. In the storage format of the table, when a node value 510 or an edge value 511 is extracted so as to meet an optional condition, or when sets of the node values 510 and/or the edge values 511 are connected together, such node value(s) and/or edge value(s) are known at a glance, which facilitates system operation.

The word, the symbol, the numerical formula, and the picture used as the node value 510 can be clarified by a classification of an information resource described in a nonpatent literature, J. Rowley, “The wisdom hierarchy: representations of the DIKW hierarchy”, Journal of Information Science, 33, 163 (2007). According to the literature, the information resource can be classified by a DIKW pyramid consisting of D (Data), I (Information), K (Knowledge), and W (Wisdom).

FIG. 6 is a schematic diagram illustrating the DIKW pyramid. In FIG. 6, D is an unsystematized figure or word, I is a figure or word systematized with some criterion, K is regularity or tendency derived from the information, and W is power that is determined or applied by a person using the knowledge. A hierarchy of the information resource is arranged from lower to upper and the amount of the information resource decreases in order of D, I, K, and W. The node value 510 in this embodiment basically represents an information resource corresponding to I or K. I or K makes the node value 510 to be human-recognizable and makes the information resource to have no personality. Consequently, even if an executor of this embodiment is not an expert having special expertise, the executor can specify a relationship between optional node values 510 relatively easily, scientifically, and objectively, and can increase understanding of the relationship.

The memory device may store the nodes for each classification or each hierarchy in each of categories of the material function, the molecular characteristic amount, and the constituent material. For example, the input device is beforehand set to be receivable of the classification or the hierarchy of the material function, the molecular characteristic amount, or the constituent material, thereby the nodes can be stored for each category or for each hierarchy based on the information received by the input device. The nodes are stored for each classification or for each hierarchy, thereby a drawing in a human-viewable form well agrees with a configuration of the memory device, which improves human understanding and reduces a load of the device required for input/output or arithmetic operation. At this time, the memory device may store the node such that one node belongs to a plurality of classifications or a plurality of hierarchies.

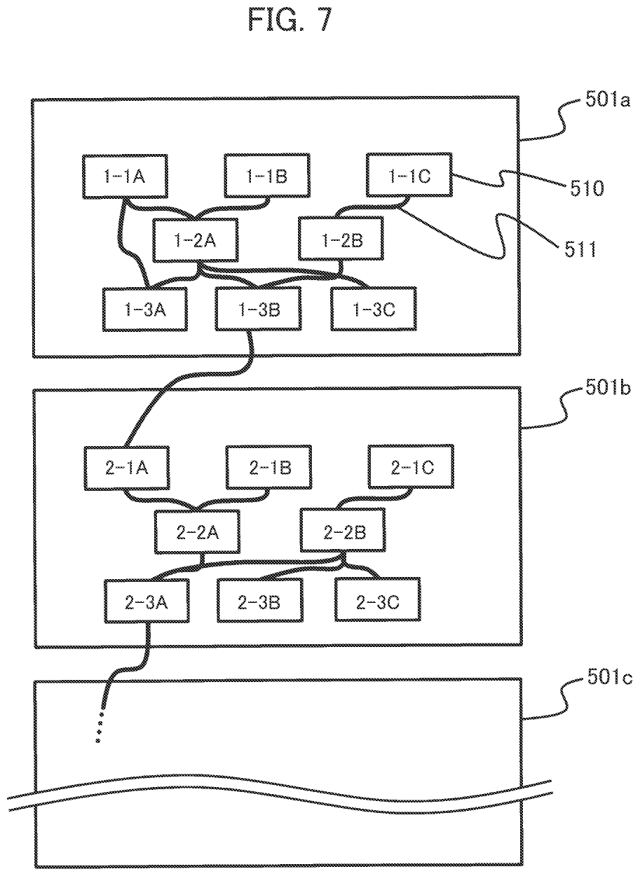

FIG. 7 is a schematic diagram illustrating an exemplary data structure of memory values stored in the memory device. The data structure of the memory values includes a set 501a of memory values of upper concept, a set 501b of memory values of medium concept, and a set 501c of memory values of lower concept. The sets of the memory values are each configured as the set 501 of the optional memory values as described above. In this embodiment, a set closer to the material function is defined as upper, and a set closer to the constituent material is defined as lower. The material function, the molecular characteristic amount, and the constituent material are categorized as upper, medium, and lower, respectively, thereby the memory values can be stored in the memory device while being maintained to be understandable by a person. In FIG. 7, each node belongs to any one of the sets, i.e., the set 501a of memory values of the upper concept, the set 501b of memory values of the medium concept, and the set 501c of memory values of the lower concept. The sets of memory values may not be of three hierarchies. The nodes 510 may be connected to each other by the edge 511 not only in each memory value set but also across two or more memory value sets.

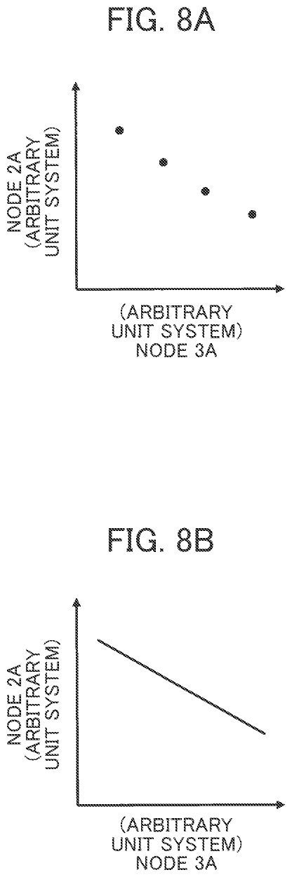

FIG. 8A illustrates an exemplary relationship between nodes. FIG. 8B illustrates another exemplary relationship between nodes. Nodes 2A and 3A connected together by an edge in FIG. 5 are now exemplified. A correlation between the nodes 2A and 3A corresponds to, for example, a case where the node 2A varies with the node 3A. In FIG. 8A, a virtual experimental result is plotted with the node 3A as input and the node 2A as output in an arbitrary unit system, and the shape of the graph suggests that the correlation shows monotone decreasing. When the correlation between the node 2A and the node 3A is thus confirmed, an edge connects between the node 2A and the node 3A.

At this time, even if the experimental result is obtained in a form of discrete values as in FIG. 8A, an interpolated relationship is desirably shown as in FIG. 8B. This is because the nodes and the edges are constituted as information resources that are easily recognized by a person as much as possible. Although the relationship between the nodes 2A and 3A cannot be determined by each discrete value, the interpolated relationship makes it possible to determine the relationship to be monotone decreasing. In light of the DIKW pyramid, it can be said that each discrete value is a numerical value systematized by experiment and thus I, and the interpolated line or the tendency of monotone decreasing is regularity derived from information and thus K.

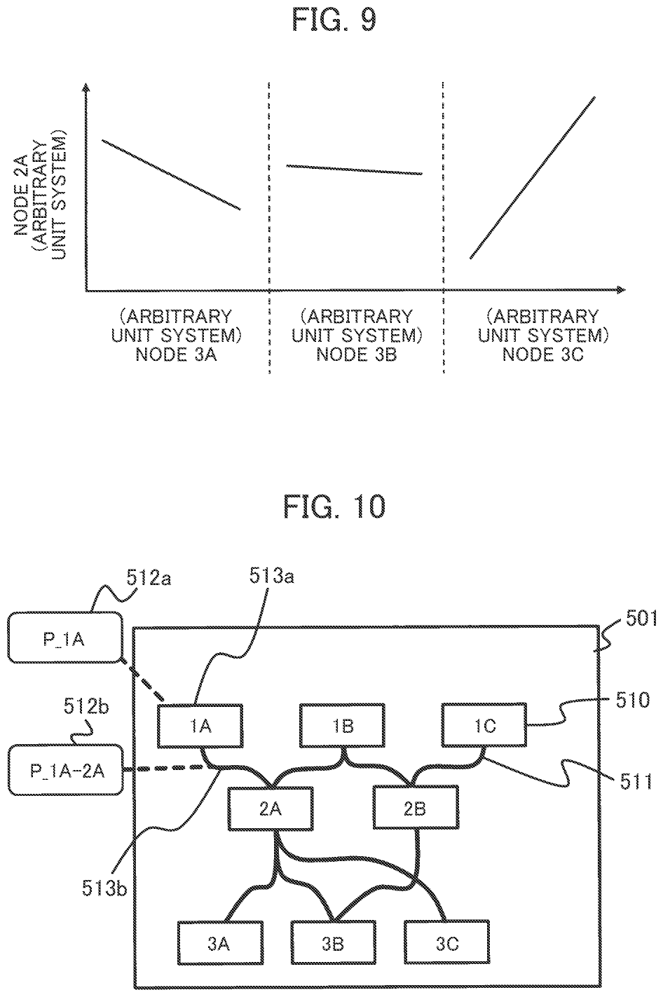

FIG. 9 illustrates exemplary relationships between the nodes 2A and 3A, between the nodes 2A and 3B, and between the nodes 2A and 3C. Although it can be read that the relationship between the nodes 2A and 3A is monotone decreasing, the relationship between the nodes 2A and 3B shows nearly-horizontal transition, and the relationship between the nodes 2A and 3C is monotone decreasing, the relationship between the nodes 2A and 3C shows a remarkable increasing tendency. In such a case, for example, decreasing or increasing and a degree of change are each represented by a numerical value and written as the edge value 511 in each of the three internodes (between the nodes 2A and 3A, between the nodes 2A and 3B, and between the nodes 2A and 3C), thereby a factorial effect for the node 2A is clarified.

An information resource is desirably added to the node value 510 or the edge value 511 as necessary. FIG. 10 is a schematic diagram illustrating an exemplary set of memory values using properties. The properties specifically indicate related matters of nodes and edges by words, symbols, numerical formulas, pictures, numerical values, or the like, and are desirably stored in the memory device together with the nodes and the edges. FIG. 10 shows a node property value 512a, a node 513a having the node property value 512a, an edge property value 512b, and an edge 513b having the edge property value 512b. Using the node property value 512a and the edge property value 512b makes it possible to describe more in detail a hierarchy to which the node belongs, the degree of a correlation indicated by the edge, and a process of investigation concerning each node or each edge.

For example, in the example of FIG. 8, a numerical formula (such as an approximate expression) of a line obtained in FIG. 8B can be stored in the property of the edge value 501 connecting between the nodes 2A and 3A. With the experiment (FIG. 8A) by which such a numerical formula has been obtained, conducting conditions, result data, a data storage site, and the like can be stored in the property.

The edge, which basically indicates the correlation, can indicate a causation by using the property. A hierarchical relationship of the nodes can be more clearly described by indicating the causation. For example, between nodes 1B and 2B in FIG. 10, when an effect of the node 1B is obtained caused by the node 2B, the node 1B can be positioned as an upper hierarchy while the node 2B as a lower hierarchy. Examples of such positioning include arrangement of the relationship between a characteristic amount hierarchy I (upper) and a characteristic amount hierarchy II (lower) in FIG. 16.

The central control unit decodes a command for each device, and sends an instruction required for executing the command to the device. Further, the central control unit can issue an instruction to the memory device to refer to a required memory value. For example, the central control unit allows an optional arithmetic unit to read an input value or a memory value and execute arithmetic operation, and stores operation results into the memory device. In addition, the central control unit instructs output of the memory value.

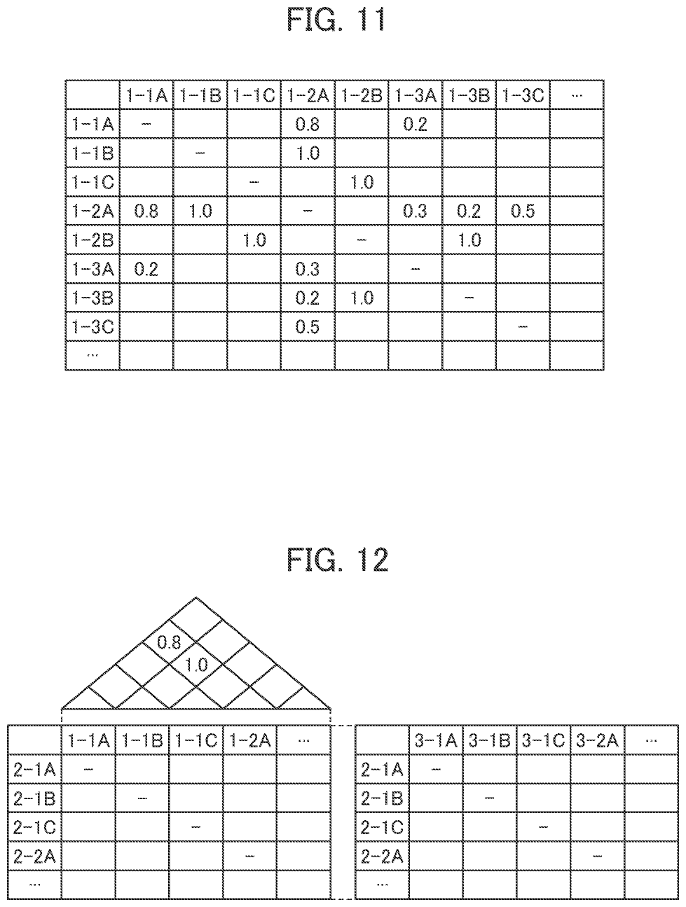

The output device includes a display, a speaker, or the like. The output device outputs a constituent material that achieves a desired material function calculated by the arithmetic unit. The output device may simultaneously output a plurality of nodes correlated with each other by at least one edge. For example, the output device may output the relationship between the nodes in a form of a matrix diagram. FIG. 11 is a matrix diagram illustrating an exemplary relationship between the nodes according to one embodiment of the invention. In the matrix diagram, optional node values 510 are allocated to rows and columns in a matrix form, and the edge value 511 is shown at each of places where the node values 510 on the rows and columns cross each other on the matrix. The relationship is output in the matrix diagram of FIG. 11, making it possible to check many node values 510 and many edge values 511 in good order on the basis of an optional node value 510.

FIG. 12 is a matrix diagram illustrating another exemplary relationship between nodes according to one embodiment of the invention. As with FIG. 11, a plurality of sets of optional memory values exist, and the respective sets are sorted with rows and columns. In the example of FIG. 12, a triangle matrix is also used to illustrate edge values 511 between nodes on a column (or a row). In this case, many node values 510 and many edge values 511 can be checked in good order on the basis of a set of optional memory values. As described above, the output device may output the relationship using two or more matrixes, or may clip and output part of the matrix.

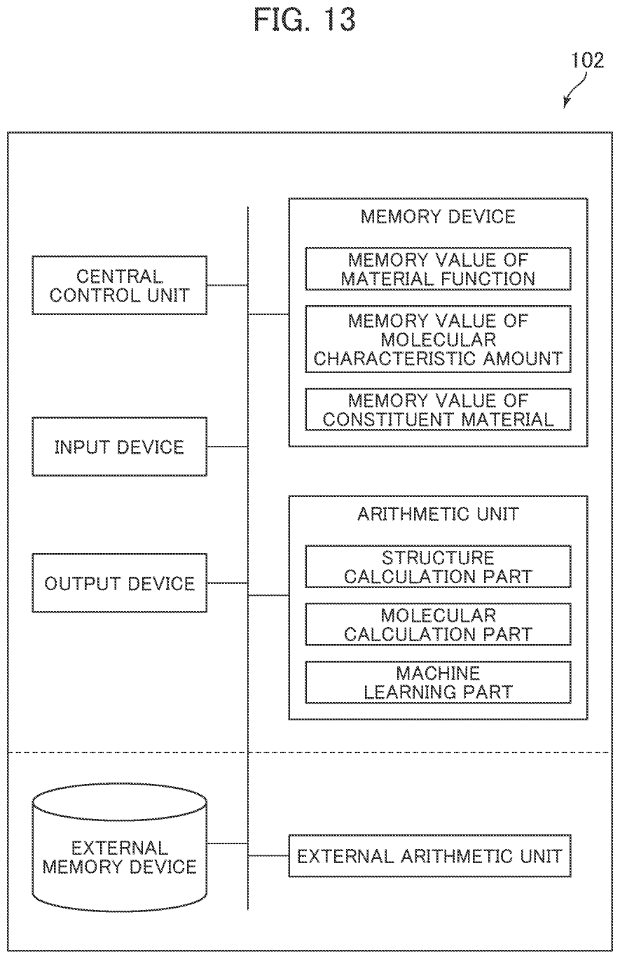

The material design system 101 may be configured to have an optional portion being separated. FIG. 13 is a schematic diagram illustrating an exemplary configuration of a material design system using an external device. In a material design system 102, part or all of the memory device and part or all of the arithmetic unit can be substituted for external devices coupled to the system 102. In other words, the material design system 102 enables storage in an external memory device, calling of externally stored data from the external memory device, external execution of various arithmetic operations, and external identification of a calculation result.

<Material Design Flow>

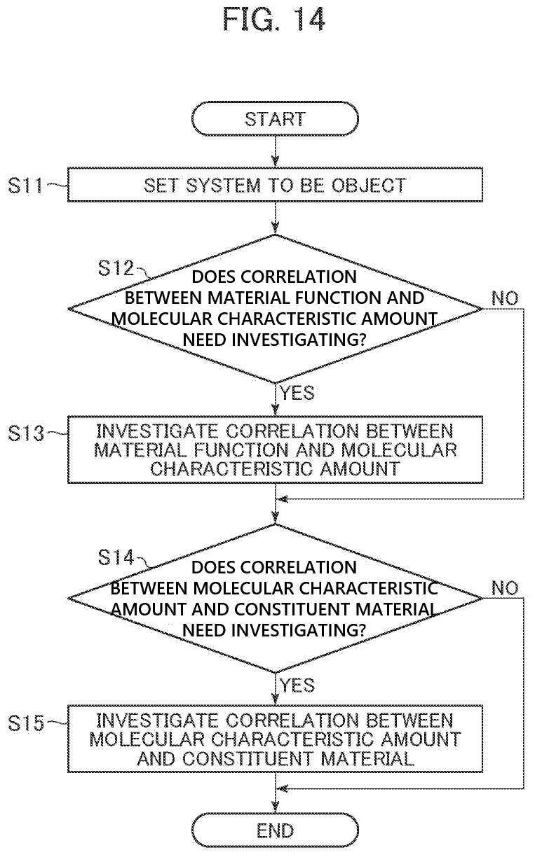

FIG. 14 is a flowchart illustrating an exemplary processing procedure of a material design system according to one embodiment of the invention. Operation based on the flowchart of FIG. 14 is as follows.

Step 11: A user sets a system to be an object. The user sets the system by entering, into the input device, investigation of correlation between a material function and molecular characteristic amount and investigation of correlation between molecular characteristic amount and a constituent material assumed in many material developments, and entering execution of such two types of investigation. The investigation of correlation described herein basically refers to clarifying presence or absence of the relevant correlation through experiment, analysis, simulation, document acquisition, and the like while focusing between two or more nodes. When the correlation is recognized, a relationship between the relevant nodes is defined (an edge is added to the relationship between the relevant nodes).

The investigation of the correlation between the material function and the molecular characteristic amount makes it possible to clarify the molecular characteristic amount directly related to a focused material function, and predict achieving means and feasibility of the material function. The investigation of the correlation between the molecular characteristic amount and the constituent material makes it possible to clarify how the focused molecular characteristic amount is achieved in a real material. The investigation of the correlation between the material function and the molecular characteristic amount and the investigation of the correlation between the molecular characteristic amount and the constituent material are successively performed, thereby it is possible to clarify how the molecular characteristic amount, which achieves the focused material function, is achieved in a real material, and thus clarify the means for achieving the material function, i.e., clarify means for producing the material.

The user may set necessity of investigation of correlation between the material functions, investigation of the correlation between the molecular characteristic amounts, and investigation of the correlation between the constituent materials. The number of nodes and the number of edges of the material function, of the molecular characteristic amount, and of the constituent material can be increased by adding the investigation of the correlation between the material functions, the investigation of the correlation between the molecular characteristic amounts, and the investigation of the correlation between the constituent materials. As a result, it is possible to increase investigation objects of the correlation between the material function and the molecular characteristic amount and investigation objects of the correlation between the molecular characteristic amount and the constituent material. Such increase in investigation objects increases possibility of finding a node having a strong correlation, which in turn increases reliability of the investigation of the correlation between the material function and the molecular characteristic amount and the investigation of the correlation between the molecular characteristic amount and the constituent material. An effect on efficiency is more expectable from increasing the number of internodes to be investigation objects than from improving accuracy of correlation between specific nodes. The reason for this is as follows. Many molecular characteristic amounts each have some correlation with an optional material function, and a few molecular characteristic amounts among those each probably have a large correlation therewith. Thus, how quickly a molecular characteristic amount having a large correlation is found is more important than accuracy of the investigation.

When parameters necessary for the investigation of the correlation between the material function and the molecular characteristic amount or the investigation of the correlation between the molecular characteristic amount and the constituent material are known, the parameters may be beforehand received by the input device in step 11.

Step 12: The arithmetic unit determines necessity of investigation of the correlation between the material function and the molecular characteristic amount based on the information received by the input device. If the investigation is necessary, the procedure is passed to step 13. If the investigation is not necessary, the procedure is passed to step 14.
Step 13: The structure calculation part of the arithmetic unit investigates the correlation between the material function and the molecular characteristic amount. In other words, the structure calculation part of the arithmetic unit calculates the molecular characteristic amount, which satisfies a desired material function, in a manner allowing expression of the molecular characteristic amount to be expressed. When the correlation between the material function and the molecular characteristic amount has been investigated, the procedure is passed to step 14.
Step 14: The arithmetic unit determines necessity of investigation of the correlation between the molecular characteristic amount and the constituent material based on the information received by the input device. If the investigation is necessary, the procedure is passed to step 15. If the investigation is not necessary, the arithmetic operation by the arithmetic unit is ended.
Step 15: The molecular calculation part of the arithmetic unit investigates the correlation between the molecular characteristic amount and the constituent material. In other words, the molecular calculation part of the arithmetic unit calculates the constituent material, which achieves the molecular characteristic amount calculated by the structure calculation part, in a manner allowing expression of the molecule itself to be expressed.

The investigation of the correlation between the material function and the molecular characteristic amount in step 13 and the investigation of the correlation between the molecular characteristic amount and the constituent material in step 15 are now described more in detail.

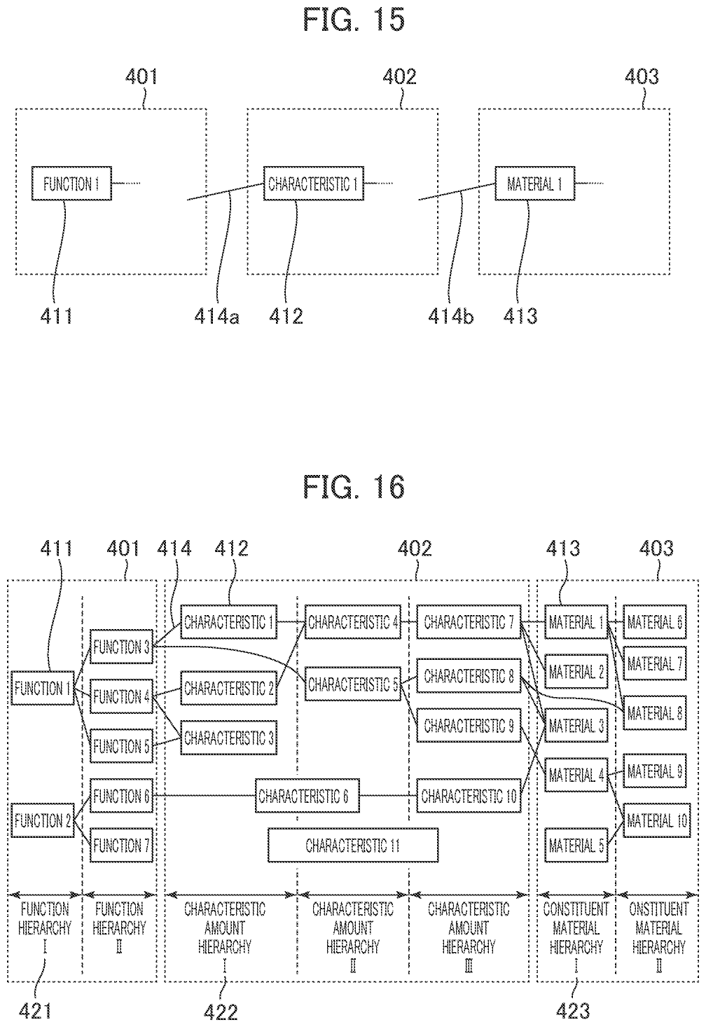

FIG. 15 is a schematic diagram illustrating in a simplified manner an exemplary relationship between the material function, the molecular characteristic amount, and the constituent material. In the figure, an edge over between the material-function node group 401 and the molecular-characteristic-amount node group 402 is defined as an edge 414a over between the material function and the molecular characteristic amount, and an edge over between the molecular-characteristic-amount node group 402 and the constituent-material node group 403 is defined as an edge 414b.

With material development, a material having a function high in market value is highly demanded. Hence, a desired material function is first determined, and then a constituent material is required so as to meet the material function. In a material design flow according to one embodiment of the invention, therefore, the correlation between the material function and the molecular characteristic amount is first investigated, and then the correlation between the molecular characteristic amount and the constituent material is investigated, thereby a material meeting a desired material function is efficiently designed. In other words, the edge 414a over between the material function and the molecular characteristic amount is first investigated, and then the edge 414b over between the molecular characteristic amount and the constituent material is investigated. The molecular characteristic amount correlating with both the material function and the constituent material is shown, thereby it is possible to clarify the principle and a mechanism of the material function, and clarify means for achieving the material function.

The respective nodes of the material function, the molecular characteristic amount, and the constituent material are desired to be defined strictly as much as possible to facilitate description of the nodes and edges and investigation of internodes. Thus, the material function node group 401, the molecular characteristic amount node group 402, and the constituent material node group 403 are each preferably classified into hierarchies, groups, or the like.

FIG. 16 is a schematic diagram illustrating an exemplary relationship between the material function, the molecular characteristic amount, and the constituent material while showing hierarchical categorization. As illustrated in FIG. 16, the hierarchy is categorized into a material function hierarchy 421, a molecular characteristic amount hierarchy 422, and a constituent material hierarchy 423. The hierarchy to be set can be defined with a matter common to two or more nodes. Examples of the matter common to two or more nodes include a time scale and a spatial scale. Specifically, nodes having similar spatial scales are attached to the same hierarchy. A relative position of each node is categorized into a hierarchy depending on its principle, connotation, extrapolation relationship, or meaning, and the node is attached to any one hierarchy, thereby investigation of an internode between adjacent hierarchies can be prioritized in the investigation of the correlation between the material function and the molecular characteristic amount and in the investigation of the correlation between the molecular characteristic amount and the constituent material. The adjacent hierarchies allow extraction of an investigation object that is directly investigable or readily investigated indirectly. Consequently, when a correlation between nodes is clarified, an edge is added (recorded) between the relevant nodes. Connection of an edge for each hierarchy allows clarification of a constituent material meeting a desired material function. This further allows adoption of the following strategy: one of the adjacent hierarchies, which is closer to the material function (on the left side of FIG. 16), is preferentially subjected to investigation. Consequently, investigation can be conducted from around a node directly connected with a desired material function, leading to efficient extraction of an investigation object.

In hierarchical categorization of a node, when a hierarchy, to which an optional node is to belong, is not clear, a user may attach the node to one reasonable hierarchy, for example. In addition, the user may attach the node to two or more reasonable hierarchies or all hierarchies.

When a hierarchy, to which an optional node is to belong, is not clear, the user preferably attaches the node to two or more reasonable hierarchies or all hierarchies. More preferably, the user attaches the node to two or more hierarchies under user determination, for example.

By attaching a node to one reasonable hierarchy, the user can operate the system as in the case where a hierarchy, to which the node is to belong, is clear. However, since priority of investigation may be determined according to the roughly determined hierarchy, an error tends to occur. In addition, when a user visualizes (outputs) a known node, erroneous recognition is likely to occur.

A node is attached to two or more hierarchies, thereby even if a hierarchy, to which the node is to belong, is not clear, a hierarchy distance between optional nodes can be measured, and investigation of between nodes of adjacent hierarchies can be prioritized. Attaching a node to two or more hierarchies helps a user to determine which hierarchy an optional node preferentially belongs to when the user visually identifies a visualized (output) node or edge. When a node is attached to two or more reasonable hierarchies, since the number of options of internodes to be candidates of investigation is small compared with a case where the node is attached to all hierarchies, an investigation object can be promptly determined. The node is preferably attached to two or more hierarchies, for example, under determination of the user in order to decrease the number of options.

Even if nodes have been categorized into hierarchies, investigation may be conducted from other than between nodes of adjacent hierarchies. For example, a user can use the input device to determine definition of the hierarchy and classification of the node. In addition, even if the user inputs no hierarchy, the system can sort the contents (such as numerical values) of a node in ascending or descending order or group the contents based on similarities between words to define the hierarchy.

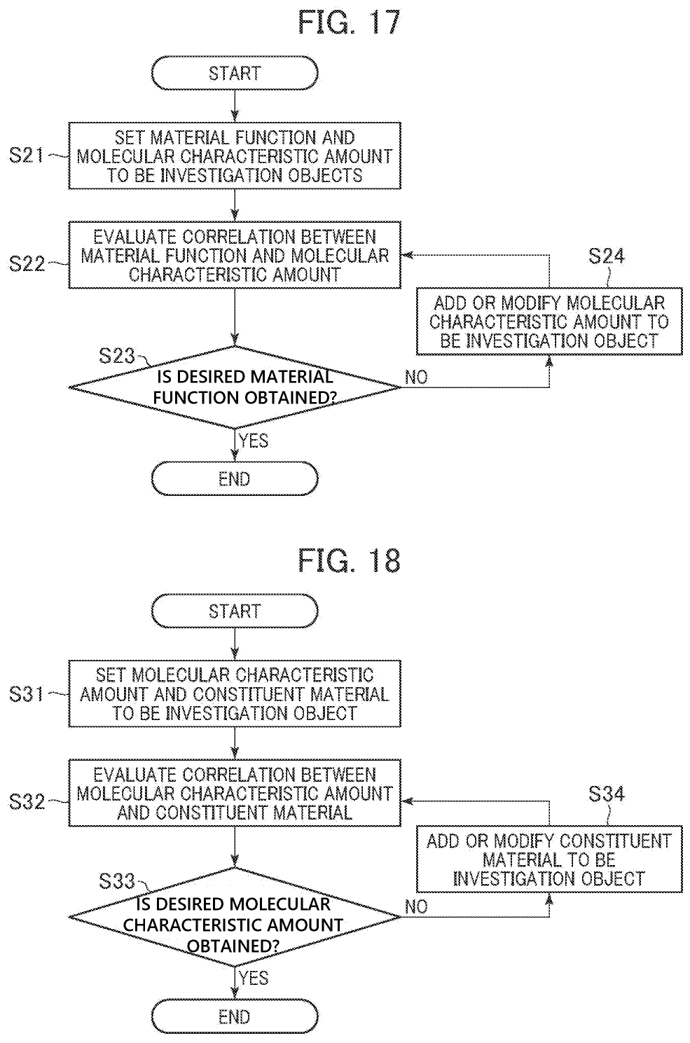

FIG. 17 is a flowchart illustrating an exemplary processing procedure of detailed investigation of the correlation between the material function and the molecular characteristic amount in step 13 of FIG. 14.

Step 21: The investigation system of the molecular characteristic amount sets in the input device a material-function node and a molecular-characteristic-amount node to be investigation objects of a user. To investigate the correlation between the material function and the molecular characteristic amount, an investigation range, if required, is set for the material function or the molecular characteristic amount. For example, when the material function and the molecular characteristic amount are each defined in an optional unit system, a numerical value range is set, if required. If setting contents and a parameter necessary for step 21 are clear at a point of step 11, the setting and the parameter necessary for step 21 can be entered at the point of step 11.
Step 22: The structure calculation part of the arithmetic unit investigates a correlation between a desired material function and optional molecular characteristic amount. In step 22, the correlation can be investigated by a method that can express the node 412 of the molecular characteristic amount. The investigation means includes experiment, analysis, simulation, and document acquisition.

In case of using experiment or analysis for investigation, reliability of an investigation result can be improved through read of a result of the experiment or the analysis, statistical analysis, machine learning operation, or search and acquisition of a similar literature. In case of using simulation, examples of a usable method, in which the node 412 of the molecular characteristic amount can be used as an input value, include the all-atom molecular dynamics method, the coarse-grained molecular dynamics method, and the dissipative particle dynamics method, which each can treat dynamic behavior of molecules, the Monte Carlo method and the Cellular Automaton which each can perform calculation using probability theory on a unit such as a molecule of a material, and the particle method, the finite element method, the finite difference method, and the finite volume method, which each can treat a macroscopic material in a subdivided unit.

The all-atom molecular dynamics method can treat a group of two or more molecules, and may use many types of known potential functions and thus easily reflects the intramolecular characteristic amount by selecting the potential function. The coarse-grained molecular dynamics method and the dissipative particle dynamics method each consider several atoms as an atom group, and thus are each high in degree of freedom at setting of the atom group, and each easily reflect both the intramolecular characteristic amount and the intermolecular characteristic amount. The Monte Carlo method and the Cellular Automaton each can define an event occurring at discrete time or probability of the event, and thus each easily reflect the intermolecular characteristic amount related to a change in coordinates of particles, including diffusion and adsorption of the particles. The particle method, the finite element method, the finite difference method, and the finite volume method each use, as input, characteristics of a material as an atomic aggregate, and thus more easily reflect macroscopic intermolecular characteristic amount than the above-described various methods. Specifically, the intermolecular characteristic amount is easily reflected by the particle method in investigation of a material, of which the large deformation should be considered, by the finite element method in investigation of a material, of which the complex shape should be considered, and by the finite difference method or the finite volume method in investigation of a material, of which the physical phenomenon of a flux should be considered.

Step 23: The arithmetic unit determines whether the molecular characteristic amount investigated in step 22 obtains a desired material function. Although any method may be used for the determination, a preferred method can make a determination to be statistically significant. For example, the following procedure is preferable: a desired material function is beforehand defined as quantitative data or qualitative data, and a particular investigation result is subjected to various tests of statistics under an optional significant level to determine significance on whether the investigation result meets the desired material function. If the desired material function is obtained, the flow of the investigation system of the molecular characteristic amount is ended. If the desired material function is not obtained, the procedure is passed to step 24.
Step 24: A/the molecular characteristic amount to be an investigation object is added or modified, and the procedure is returned to step 22 and the correlation between the material function and the molecular characteristic amount is investigated again. For example, the molecular characteristic amount can be added or modified through user input into the input device. The investigation is repeated until the desired material function is obtained. As a result, a molecular characteristic amount correlating with the desired material function is obtained.

FIG. 18 is a flowchart illustrating an exemplary processing procedure of detailed investigation of the correlation between the molecular characteristic amount and the constituent material.

Step 31: A molecular characteristic amount and a constituent material to be investigation objects are set. As the molecular characteristic amount, a molecular characteristic amount obtained by the structure calculation part of the arithmetic unit (hereinafter, referred to as desired molecular characteristic amount) can be used. The constituent material can be set through user input into the input device.
Step 32: The molecular calculation part of the arithmetic unit investigates a correlation between the molecular characteristic amount and the constituent material. In step 32, the correlation can be investigated by a method that can express the node 413 of the constituent material. The investigation method is the same as in step 22. In step 32, for example, a usable method for simulation, in which a node 33 of the constituent material can be used as an input value, include the all-atom molecular dynamics method that can calculate dynamic behavior of molecules, the empirical molecular orbital method, the first principle molecular dynamics method (including the Car-Parrinello method), and the static first principle calculation that can calculate an electronic state, such as the molecular orbital method and the density-functional approach.

The all-atom molecular dynamics method can perform comparatively large-scale dynamic calculation while treating each atom, and can investigate diffusion, viscosity, and dynamic characteristics of molecules. The static first principle calculation can accurately investigate static characteristics of a material, such as the most stable structure, various energy values, and phonon. The first principle molecular dynamics method can perform calculation of dynamics in consideration of a chemical reaction, and can investigate, for example, a chemical reaction process in consideration of temperature and atom migration.

Step 33: The arithmetic unit determines whether the constituent material investigated in step 32 obtains a desired molecular characteristic amount. The same method as in step 23 can be used for the determination. If the desired molecular characteristic amount is obtained, the investigation flow of the correlation between the molecular characteristic amount and the constituent material is ended. If the desired material function is not obtained, the procedure is passed to step 34.
Step 34: A/the constituent material to be an investigation object is added or modified, and the procedure is returned to step 32 and the correlation between the molecular characteristic amount and the constituent material is investigated again. For example, the constituent material can be added or modified through user input into the input device. The investigation is repeated until the desired molecular characteristic amount is obtained. As a result, a constituent material correlating with the desired molecular characteristic amount is obtained.

As described above, the material design system according to one embodiment of the invention investigates the correlation between the material function and the molecular characteristic amount and then investigates the correlation between the molecular characteristic amount and the constituent material, thereby can efficiently obtain the molecular characteristic amount and the constituent material, which each correlate with the desired material function.

### EXAMPLES

Example of material design using the above-described material design system is described below. Material development is now assumed so as to design a polymeric material meeting optional mechanical properties. A polymeric material forming a spherulite is given as an example.

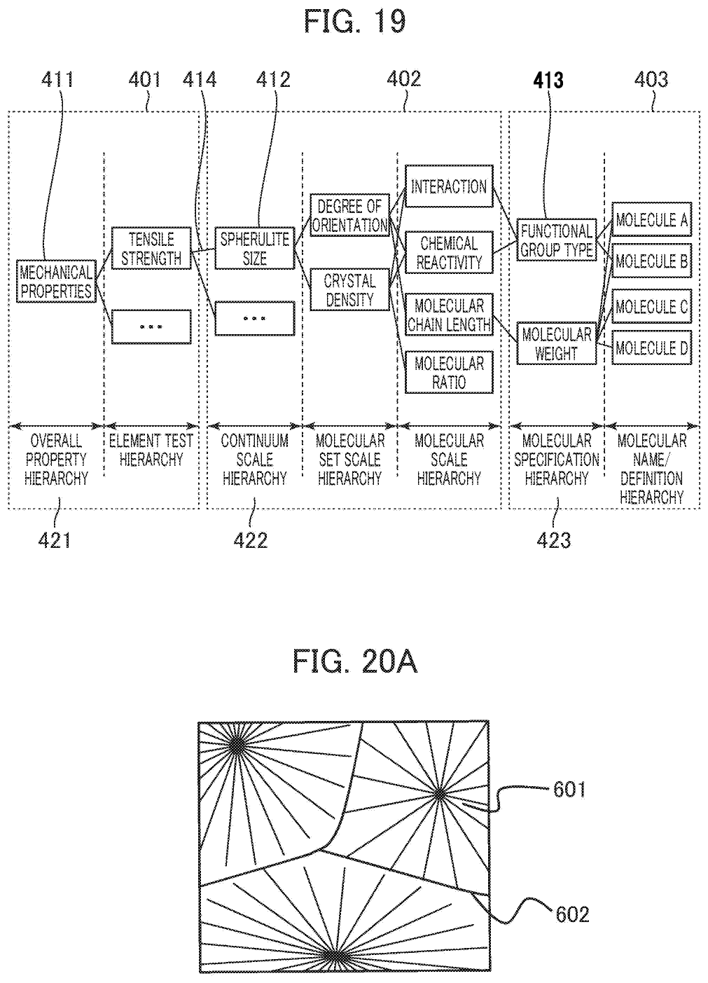

FIG. 19 is a schematic diagram illustrating an exemplary relationship between a material function, molecular characteristic amount, and a constituent material for a polymeric material. FIG. 19 illustrates a correlation between the material function 401, the molecular characteristic amount 402, and the constituent material 403. Specifically, FIG. 19 illustrates the node 411 of the material function such as mechanical properties, the node 412 of the molecular characteristic amount such as spherulite size, the node 413 of the constituent material such as a type of a functional group, and the edges 414 connecting the nodes to each other. Such nodes are categorized into several hierarchies. FIG. 19 shows the material function hierarchy 421 such as an overall characteristic hierarchy, the molecular characteristic amount hierarchy 422 such as a continuum scale hierarchy, and the constituent material hierarchy 423 such as a molecular specification hierarchy.

The nodes, the edges, and the hierarchical structure may be initially given as input, or may be added or modified in the middle of the following procedure. In this case, the nodes and the hierarchical structure are assumed to be initially given. Although no edge is shown in principle if no investigation result exist, edges are assumed to be added in the middle of the following procedure in this embodiment.

The mechanical properties are determined by tensile strength, for example. Hence, FIG. 19 shows a node of tensile strength in a hierarchy of an element test of the material function. In this Example, material design meeting a desired material function is performed with the molecular characteristic amount as a start point. Hence, the node of the molecular characteristic amount should be first focused. In various molecular characteristic amounts, a node considered to be in the continuum scale hierarchy includes a spherulite size node, a node considered to be in a molecular association scale hierarchy includes a node of the degree of orientation and a crystal density node, and a node considered to be in a molecular scale hierarchy includes an interaction node and a chemical reactivity node. For investigation of the correlation with the node of tensile strength, a node in the nearest scale hierarchy is the spherulite size node.

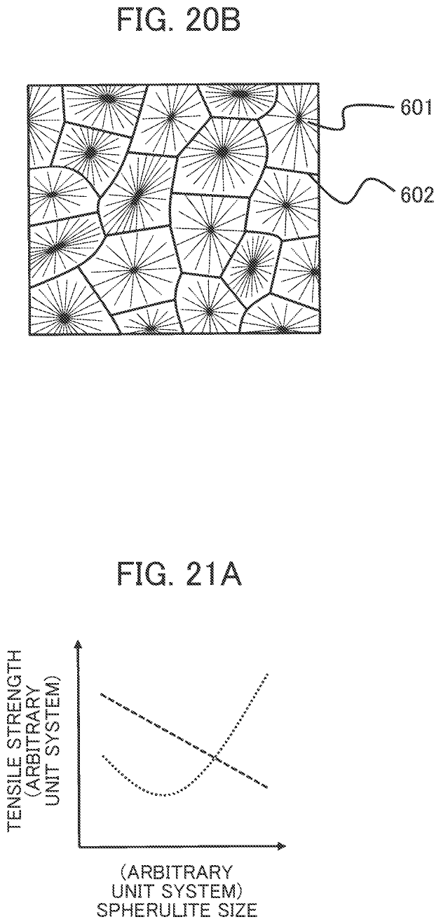

FIG. 20A is a schematic diagram illustrating an exemplary spherulite of a polymeric material. FIG. 20B is a schematic diagram illustrating another exemplary spherulite of a polymeric material. FIG. 20A and FIG. 20B are schematic diagrams of materials having different spherulite sizes. FIG. 20A and FIG. 20B each show optional spherulite 601 and an optional spherulite boundary 602. Radially extending lines in the optional spherulite 601 each indicate a crystal direction.

In this Example, a spherulite size, probably closest to the desired material function, is first investigated. In this case, tensile strength can be calculated using the finite element method or the coarse-grained molecular dynamics method, which can express a shape and a structure of spherulite.

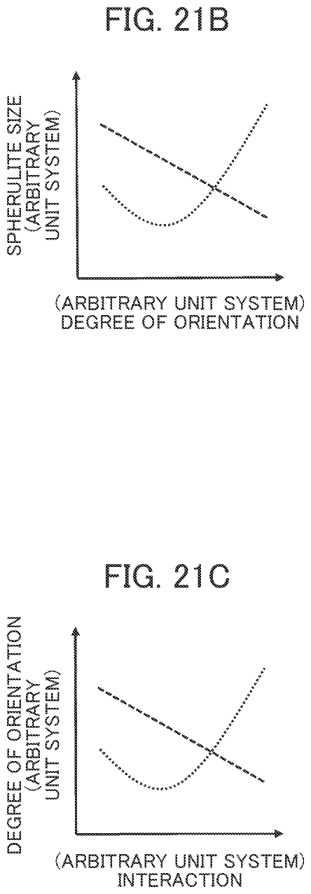

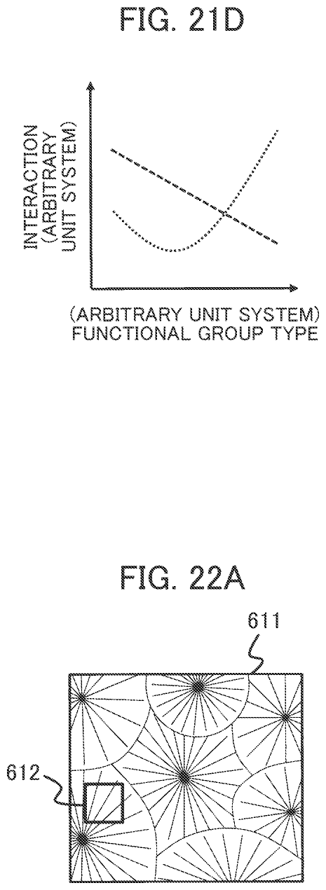

FIG. 21A illustrates an exemplary correlation between spherulite size and tensile strength as nodes. FIG. 21A shows a result of determining tensile strength using the finite element method or the coarse-grained molecular dynamics method with spherulite size as an input value. In general, such an investigation result probably includes monotone increasing, monotone decreasing, and repeated increase and decrease. FIG. 21A shows an aspect where the result is obtained in a form of a monotone decreasing line or a downward convex quadratic curve. Such investigation results enable control of tensile strength using spherulite size. When some correlation is seen, the arithmetic unit sends an edge into between the relevant nodes.

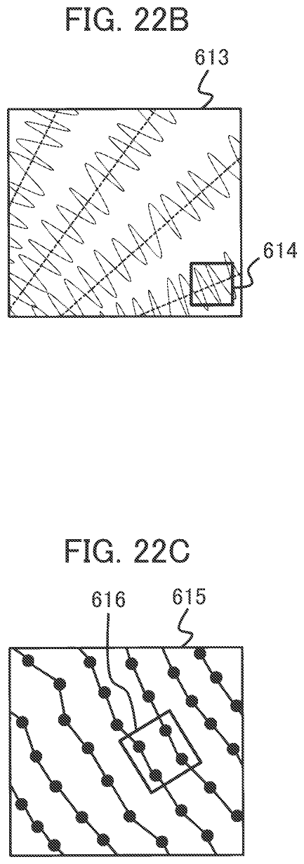

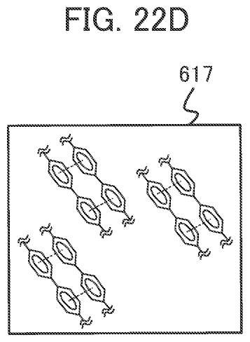

FIG. 22A is a schematic diagram to be a base of the investigation of the relationship between the tensile strength and the spherulite size in FIG. 21A. In FIG. 22A, the spherulite size is expressed by an element-test-scale sectional view 611 of a spherulite material. The arithmetic unit can investigate tensile strength by using such a calculation model.

Subsequently, investigation is made on a node that is considered to be closest to the spherulite size node, of which the influence on the desired material function has been clarified. In FIG. 19, a node of the degree of orientation is one of the nodes, for each of which the scale hierarchy is closest to that for the spherulite size node. Herein, the spherulite size can be calculated from the degree of orientation using, for example, the coarse-grained molecular dynamics method that can express a higher-order structure of polymer.

FIG. 21B illustrates an exemplary correlation between the degree of orientation and spherulite size as nodes. FIG. 21B shows that as a result of determining the spherulite size with the degree of orientation as an input value, a monotone decreasing line or a downward convex quadratic curve is obtained. Such an investigation result enables control of the spherulite size using the degree of orientation. When some correlation is seen, an edge is sent into between the relevant nodes.

FIG. 22B is a schematic diagram to be a base of the investigation of the relationship between the spherulite size and the degree of orientation in FIG. 21B. FIG. 22B corresponds to a partial enlargement of a local portion 612 of an element-test-scale section of the spherulite material of FIG. 22A. In a continuum-scale sectional view 613 of the spherulite material, the meandering solid line indicates an aspect of a folded polymer, and a broken line indicates a crystal direction of the spherulite. FIG. 22B shows orientation of the polymer. Such a calculation model can be used to investigate the spherulite size.

According to this and previous investigation, tensile strength can be controlled with the degree of orientation as an input item by means of the spherulite size.

Subsequently, investigation is made on a node that is considered to be closest to a node of the degree of orientation, of which the influence on the desired material function has been clarified. In FIG. 19, a node of interaction is one of the nodes, for each of which the scale hierarchy is closest to that for the node of the degree of orientation. Herein, the interaction is assumed to be physical interaction such as Van der Waals' forces. The degree of orientation can be calculated from the interaction using the coarse-grained molecular dynamics method or the all-atom molecular dynamics method, which can express a chemical structure of a molecule.

FIG. 21C illustrates an exemplary correlation between interaction and the degree of orientation as nodes. FIG. 21C shows that as a result of determining the degree of orientation with the interaction as an input value, a monotone decreasing line or a downward convex quadratic curve is obtained. Such an investigation result enables control of the degree of orientation using the interaction. When some correlation is seen, the memory device sends an edge into between the relevant nodes.

FIG. 22C is a schematic diagram to be a base of the investigation of the relationship between the degree of orientation and the interaction in FIG. 21C. FIG. 22C corresponds to a partial enlargement of a local portion 614 of a continuum-scale section of the spherulite material of FIG. 22B. In a molecular-association-scale sectional view 615 of the spherulite material, a dot indicates an atom or atomic group consisting of several atoms, and a solid line indicates binding within a polymer. FIG. 22C shows molecular structures and interaction therebetween. The arithmetic unit can investigate the degree of orientation using such a calculation model.

According to this and previous investigation, tensile strength can be controlled with the interaction as an input item by means of the spherulite size and the degree of orientation.

Subsequently, investigation is made on a node that is considered to be closest to an interaction node, of which the influence on the desired material function has been clarified. In FIG. 19, a node of a functional group type is one of the nodes closest to the interaction node. The interaction can be calculated from the functional group type using the all-atom molecular dynamics method or the molecular orbital method, which can express a chemical structure of a molecule.

FIG. 21D illustrates an exemplary correlation between a functional group type and interaction as nodes. FIG. 21D shows that as a result of determining the interaction with the functional group type as an input value, a monotone decreasing line or a downward convex quadratic curve is obtained. Such an investigation result enables control of the interaction using the functional group type. When some correlation is seen, the arithmetic unit sends an edge into between the relevant nodes.

FIG. 22D is a schematic diagram to be a base of the investigation of the relationship between the interaction and the functional group type in FIG. 21D. FIG. 22D corresponds to a partial enlargement of a local portion 616 of a molecular-association-scale section of the spherulite material of FIG. 22C. A partial intramolecular chemical structure is shown in a molecular scale sectional view 617 of an optional spherulite material. The arithmetic unit can investigate the interaction using such a calculation model.

According to this and previous investigation, tensile strength can be controlled with the functional group type as an input item by means of the spherulite size, the degree of orientation, and the interaction. Since the functional group type is uniquely determined from a molecule itself (such as molecule A or B in FIG. 19), a molecule can be selected so as to achieve a desired tensile strength.

The following shows effects of this Example that are not described above.

According to this Example, the material function, the molecular characteristic amount, the constituent material, and relationships between those are sorted out, and thus a guideline for material design can be found objectively and rationally. Hence, a user of this Example need not necessarily have specialty on an optional technical field, making it possible to reduce a user load. In addition, the material function, the molecular characteristic amount, the constituent material, and a relationship between them are sorted out, which makes the principle or logic of a mechanism to be clear and thus can improve the authenticity of each content.

In addition, the range and necessity of investigation are clarified, thereby unnecessary restrictions on use situation and use environment can be removed, making it possible to reduce a load on environment creation. For example, it can be determined that equipment for experiment, analysis, and/or simulation is prepared only for a portion necessary to be investigated.

With information and knowledges (for example, the reason for using a relevant technique, a technical device, and interpretation or findings after a result is obtained) that are personally owned in the background art, this Example can provide a function of storing and visualizing such information and knowledges. The function therefore can be used for storing, systematizing, and transmitting the information and knowledges of a user of this Example, and besides can be used for learning and promotion of understanding by the user, and provision of opportunities of experience accumulation.

### LIST OF REFERENCE SIGNS

101 . . . Material design system;
102 . . . Material design system using external device;
201 . . . Output screen; 202 . . . Entry field; 401 . . . Material-function node group;
402 . . . Molecular-characteristic-amount node group;
403 . . . Constituent-material node group;
411 . . . Material function node;
412 . . . Molecular-characteristic-amount node;
413 . . . Constituent material node;
414 . . . edge;
414a . . . Edge over between material function and molecular characteristic amount;
414b . . . Edge over between molecular characteristic amount and constituent material;
421 . . . Material function hierarchy;
422 . . . Molecular-characteristic-amount hierarchy;
423 . . . Constituent material hierarchy;
501 . . . Memory value set;
501a . . . Upper-concept memory-value set;
501b . . . Medium-concept memory-value set;
501c . . . Lower-concept memory-value set;
510 . . . Node value;
511 . . . Edge value;
512a . . . Node property value;
512b . . . Edge Property value;
513a . . . Node having node property value 612a;
513b . . . Node having node property value 612b;
601 . . . Optional spherulite;
602 . . . Optional spherulite boundary;
611 . . . Element-test-scale sectional view of spherulite material;
612 . . . Local portion of element-test-scale section of spherulite material;
613 . . . Continuum-scale sectional view of spherulite material;
614 . . . Local portion of continuum-scale section of spherulite material;
615 . . . Molecular-association-scale sectional view of spherulite material;
616 . . . Local portion of molecular-association-scale section of spherulite material; and
617 . . . Molecular-scale sectional view of spherulite material.

1. A material design system that designs a material capable of achieving a desired material function, the system comprising:
an input device receiving the desired material function; and an arithmetic unit calculating a constitutional material,
wherein the arithmetic unit includes a structure calculation part that calculates a molecular characteristic amount meeting the desired material function using a method capable of expressing molecular characteristic amount exhibited by a set of two or more atoms or molecules, and a molecular calculation part that calculates the constituent material, the constituent material achieving the molecular characteristic amount calculated by the structure calculation part, using a method capable of expressing a molecule itself.

2. The material design system according to claim 1, wherein the method capable of expressing the molecular characteristic amount includes any one of methods of an all-atom molecular dynamics method, a coarse-grained molecular dynamics method, a dissipative particle dynamics method, a Monte Carlo method, Cellular Automaton, a particle method, a finite element method, a finite difference method, and a finite volume method.

3. The material design system according to claim 1, wherein the method capable of expressing a molecule itself includes any one of methods of the all-atom molecular dynamics method, an empirical molecular orbital method, a static first principle calculation method, and a first principle molecular dynamics method.

4. The material design system according to claim 1, further comprising a memory device that stores as nodes the material function, the molecular characteristic amount, and the constituent material and stores as edges relations between the nodes based on a calculation result of the structure calculation part and a calculation result of the molecular calculation part.

5. The material design system according to claim 4, wherein the memory device stores contents of the nodes and contents of the edges in a form of a table or a graph.

6. The material design system according to claim 4, wherein the memory device stores the nodes for each classification or for each hierarchy in each of categories of the material function, the molecular characteristic amount, and the constituent material.

7. The material design system according to claim 6, wherein the memory device stores the nodes while each node belongs to a plurality of classifications or a plurality of hierarchies.

8. The material design system according to claim 1, wherein the arithmetic unit includes a machine learning part executing machine learning operation, and
the structure calculation part and the molecular calculation part calculate the molecular characteristic amount and the constituent material, respectively, using operation results of the machine learning part.

9. The material design system according to claim 1, wherein the arithmetic unit determines significance of whether the molecular characteristic amount calculated by the structure calculation part meets the desired material function, and if no significance is determined, the structure calculation part calculates again a molecular characteristic amount that meets the desired material function, and
determines significance of whether the constituent material calculated by the molecular calculation part meets the molecular characteristic amount calculated by the structure calculation part, and if no significance is determined, the molecular calculation part calculates again a constituent material that achieves the molecular characteristic amount calculated by the structure calculation part.

10. The material design system according to claim 1, wherein the input device is capable of receiving as nodes the material function, the molecular characteristic amount, and the constituent material and receiving as at least one edge a relationship between two optional nodes.

11. The material design system according to claim 10, wherein the input device is capable of receiving hierarchies or classifications of the material function, the molecular characteristic amount, and the constituent material.

12. The material design system according to claim 4, further comprising an output device that outputs the constituent material calculated by the arithmetic unit,
wherein the output device outputs a plurality of the nodes correlated with each other by the edge.

13. A material design method for designing a material capable of achieving a desired material function,
wherein molecular characteristic amount that meets the desired material function is calculated using a method capable of expressing molecular characteristic amount exhibited by a set of two or more atoms or molecules, and then
the constituent material achieving the calculated molecular characteristic amount is obtained using a method capable of expressing a molecule itself.

14. The material design method according to claim 13, wherein the method capable of expressing the molecular characteristic amount includes any one of methods of an all-atom molecular dynamics method, a coarse-grained molecular dynamics method, a dissipative particle dynamics method, a Monte Carlo method, Cellular Automaton, a particle method, a finite element method, a finite difference method, and a finite volume method.

15. The material design method according to claim 14, wherein the method capable of expressing a molecule itself includes any one of methods of the all-atom molecular dynamics method, an empirical molecular orbital method, a static first principle calculation method, and a first principle molecular dynamics method.
